(19) United States
(12) Patent Application Publication
(10) Pub. No.: US 2019/0017989 A1
(43) Pub. Date: Jan. 17, 2019

(54) Tunnel Recognition Technology-Based Nano-Detection Device And Method

(71) Applicant: Wuhan University Of Science And Technology, WUHAN (CN)

(72) Inventors: SHUAI CHANG, WUHAN (CN); JIN HE, WUHAN (CN); FENG LIANG, WUHAN (CN); MINGZHU HUANG, WUHAN (CN); HUAZHI GU, WUHAN (CN)

(21) Appl. No.: 16/129,809

(22) Filed: Sep. 13, 2018

Related U.S. Application Data
(63) Continuation of application No. PCT/CN2017/089972, filed on Jun. 26, 2017.

(51) Int. Cl. G01N 33/487; G01N 27/447

(52) U.S. Cl. CPC G01N 33/48721; G01N 27/44791

(57) ABSTRACT

The present invention relates to a tunnel recognition technology-based nano-detection device and method. The said detection device consists of nano wand, plane electrode, active power and current tester. The said detection method consists of the following steps: Place the said nano wand into the test solution with a specific DNA or RNA sequence, and place the said plane electrode on the surface of the said test solution; import the said DNA or RNA sequence into the said transmission pipeline; detect and record the current change displayed by the said current tester; read the base signal of the said DNA or RNA sequence based on the said current change detected. The technical scheme of the present invention is based on DNA sequence direct reading techniques, characterized by quick reading and high accuracy.

### CROSS-REFERENCE TO RELATED APPLICATION

The present application is a continuation-application of International Application PCT/CN20171089972, with an international filing date of Jun. 26, 2017, the contents of all of which are hereby incorporated by reference.

### BACKGROUND

### 1. Technical Field

The present invention relates to the gene detection field, and, more specifically, to a tunnel recognition technology-based nano-detection device and method.

### 2. Description of Related Art

DNA and RNW sequences are referred to as life codes. Nowadays DNA sequencing techniques are still expensive and developing slowly, and, with the aid of fluorescence or chemiluminescent substances, they are carried out indirectly through reading the optical signal released in the process of base connection to DNA chains by polymerase or ligase. The sequencing process consumes expensive and complicated optical detection systems, enzymes, biochemical reagents and consumables, and makes it difficult to lower the detection cost. In addition, high throughput sequencing techniques all conducts sequencing on amplified artifacts, and the amplification reaction process not only is time-consuming and expensive, but also inevitably produces amplification bias and erases the modification and other relevant information of the original sequences in the amplification process. Thus, it's highly desirable to develop a DNA sequence direct reading techniques that require no complicated biological reagent or optical detection system.

For the purpose of directly reading the single bases of long DNA chains and their sequences, in the past decade many new sequencing methods have been proposed, which, however, are not very ideal in terms of reading velocity and accuracy.

### SUMMARY

In order to increase the reading velocity and accuracy of DNA and RNA base sequences, the present invention provides a tunnel recognition technology-based nano-detection device and method.

On the one hand, the present invention provides a tunnel recognition technology-based nano-detection device, wherein, the said detection device consists of nano wand, plane electrode, active power and current tester; the said nano wand is made through double-bore quartz tube drawing; one bore of the said double-bore quartz tube is made into an electrode through electrode material filling, while the other bore serves as the transmission pipeline; one end of the said electrode is connected to one end of the current tester, while the other end of the said electrode is connected to the active power; electrical connection is provided between the said plane electrode and the other end of the said current tester.

Furthermore, the two bores of the said double-bore quartz tube have the same inner diameter ranging between 10 and 100 nm, and the bore edge distance of the said two bores ranges between 1 and 10 nm.

Furthermore, the said inner diameter is 50 nm, and the said bore edge distance is 2.5 nm.

According to one aspect of the present invention, the said electrode is a carbon electrode.

Furthermore, the said electrode and the said plane electrode surface are decorated with recognition molecules, and the said recognition molecules are connected to the said electrode and the said plane electrode surface via trimethyl.

According to one aspect of the present invention, the said carbon electrode can be replaced by a gold electrode or palladium electrode, or the said carbon electrode surface can be electroplated with a layer of gold or palladium film.

Furthermore, the said current tester is connected to a host computer or other detection systems.

Furthermore, the said electrode and the said plane electrode surface are decorated with recognition molecules, and the said recognition molecules are connected to the said electrode and the said plane electrode surface via mercapto group.

On the other hand, the present invention also provides a tunnel recognition technology-based nano-detection method, wherein, the said detection method consists of the following steps:

Place the said nano wand used on the said nano-detection device into the test solution with a specific DNA or RNA sequence, place the said plane electrode on the surface of the said test solution;

Import the said DNA or RNA sequence into the said transmission pipeline;

Detect and record the current change displayed by the said current tester;

Read the base signal of the said DNA or RNA sequence based on the said current change detected.

Furthermore, in the said step of importing the said DNA or RNA sequence into the said transmission pipeline, the transmission velocity of the said DNA or RNA sequence is changed through changing the said test solution's salt concentration gradient, electric field and external pressure.

When the DNA or RNA sequence in the test solution passes through the transmission pipeline of the said detection device based on the above detection device and method, it will give rise to change in the tunnel current between the nano wand electrode and the plane electrode; relying on the detection and records of the current tester, the corresponding signal of the related DNA or RNA base can be read. This direct sequencing device and method have the technical effects of quick detection and high accuracy.

### BRIEF DESCRIPTION OF THE DRAWINGS

To more clearly describe the technical scheme of the present invention's examples or existing technologies, the drawings to be used in the description of the examples or existing technologies will be introduced one by one below. Apparently, the drawings described below provide some examples of the present invention, and, ordinary technical staff of this field can also obtain other drawings based on these drawings without contributing creative labor.

FIG. 1 depicts the structural schematic diagram of a tunnel recognition technology-based nano-detection device.

FIG. 2 depicts the structural schematic diagram of the nano wand.

FIG. 3 provides the flow diagram of a tunnel recognition technology-based nano-detection method.

### DETAILED DESCRIPTION

The principles and characteristics of the present invention are described on the basis of these drawings; the examples cited are provided only to interpret the present invention, not to limit its scope.

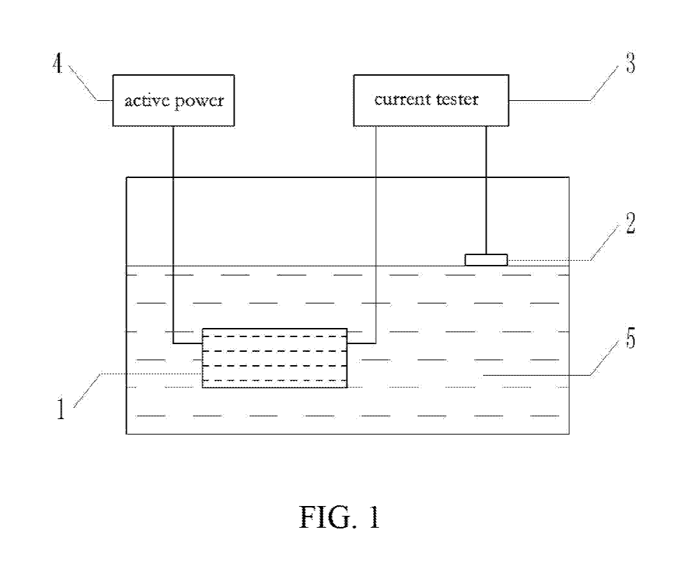

FIG. 1 depicts the structural schematic diagram of a tunnel recognition technology-based nano-detection device. As shown in FIG. 1, the said detection device consists of nano wand 1, plane electrode 2, current tester 3 and active power 4, wherein, the said nano wand 1 is made through double-bore quartz tube drawing; one bore of the said double-bore quartz tube is made into an electrode 11 through electrode material filling, while the other bore serves as the transmission pipeline 12; one end of the said electrode 11 is connected to one end of the current tester 3, while the other end of the said electrode 11 is connected to the active power 4; electrical connection is provided between the said plane electrode 2 and the other end of the said current tester 4.

When adopting the technical scheme of inlaying the nanoscale electrode into a nanopore or nanochannel, it's necessary prepare nanoscale through-holes, however, the success rate of direct nanoscale through-hole drilling is extremely low. On that account, the present invention adopts the method of drawing the double-bore quartz tube to prepare the nano wand, and, compared with traditional drilling methods, this method has a higher success rate.

The nano double-bore quartz tube is prepared by the following method: First use Piranha to clean the commercially purchased quartz Theta capillary tube, and then use deionized water for repeated cleaning; after that, place it in an oven at 120° C. for several hours. Several hours later, use a microelectrode tensiometer to draw the nano double-bore quartz tube, and control the head bore diameter of the nano double-bore quartz tube through adjusting the parameters of the microelectrode tensiometer, including temperature, velocity, etc. Later the optical microscope and scanning electron microscope are used to characterize the tube orifice shape and size of the nano double-bore quartz tube.

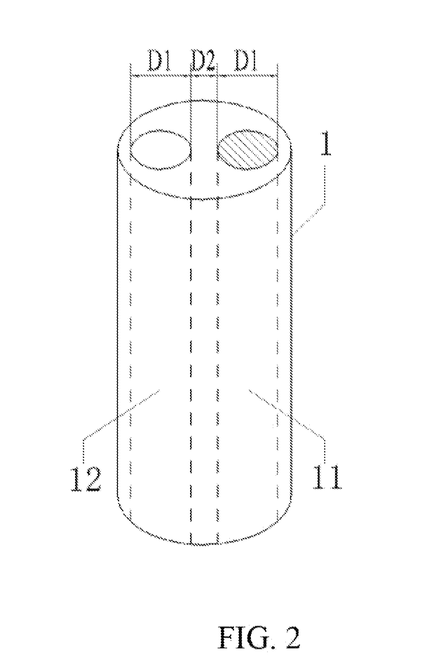

FIG. 2 depicts the structural schematic diagram of the nano wand 1. As shown in FIG. 2, the said nano wand 1 has two bores of the same inner diameter (inner diameter=D1), and the bore edge distance of the two bores is D2. D1 ranges between 10 nm and 100 nm; D2 ranges between 1 nm and 10 nm.

In one preferred example of the present invention, D1 is 50 nm, and D2 is 2.5 nm.

In one example of the present invention, the electrode 11 of the said nano wand 1 is a carbon electrode. The said carbon electrode is prepared by the following method: Use a removable rubber plug to block one bore at the end of the nano double-bore quartz tube, and then lead 25 kPa butane into the other bore. Use flames to heat the tip of the nano double-bore quartz tube for 30-40 s, after which the butane deposits on the inner wall of the quartz tube and forms stable nano carbon electrode. In the heating process, 0.5 kPa argon flow is applied around the tip of the quartz tube, with the purpose of preventing both the oxidization of the nano carbon electrode in the formation process and the deformation of the tip under high temperature.

In another example of the present invention, the said electrode 11 is a gold electrode or palladium electrode.

In yet another example of the present invention, the vacuum coating technology is employed to electroplate the carbon electrode surface with a layer of gold or palladium film.

In the chemical modification to an electrode, the chemical modification method is adopted to conduct molecular design on the electrode surface, so as to fix molecules, ions and polymers with excellent chemical properties on the electrode surface, create a special microstructure and endow the electrode with specific chemical and electrochemical properties. In this way, it makes convenience for performing the desired reaction in a high-selectivity manner, and creates unique priorities in terms of selectivity and sensitivity.

Relying on the multiple available potential fields provided by the microstructure on the surface of the chemically modified electrode, the determinand is effectively separated and enriched, and the selectivity is further increased through electrode potential control; meanwhile, the sensitivity of the measurement method and the selectivity of the modifier's chemical reaction are combined to provide an ideal three-in-one system (i.e., separation, enrichment and selectivity).

In one example of the present invention, the carbon electrode is modified, and the recognition molecules are universal recognition molecules.

In one example of the present invention, the functional groups with recognition molecules are modified, and connected to the carbon electrode via trimethyl.

In one example of the present invention, functional groups are adjusted, and increased by the length of one carbon atom to increase the degree of freedom of recognition molecules.

In one example of the present invention, the gold electrode or palladium electrode is modified, and the recognition molecules are universal recognition molecules.

In one example of the present invention, the recognition molecules are connected to the carbon electrode via mercapto group.

In one example of the present invention, the functional groups with recognition molecules are modified, and connected to the carbon electrode via trimethyl.

In one example of the present invention, the functional groups with recognition molecules are modified, and the end group is added with one carbon atom to increase the molecular length, so that the carbon atom is directly connected with the metal electrode to enhance molecular electrical conductivity.

In one example of the present invention, the said current tester is connected to a host computer or other detection systems.

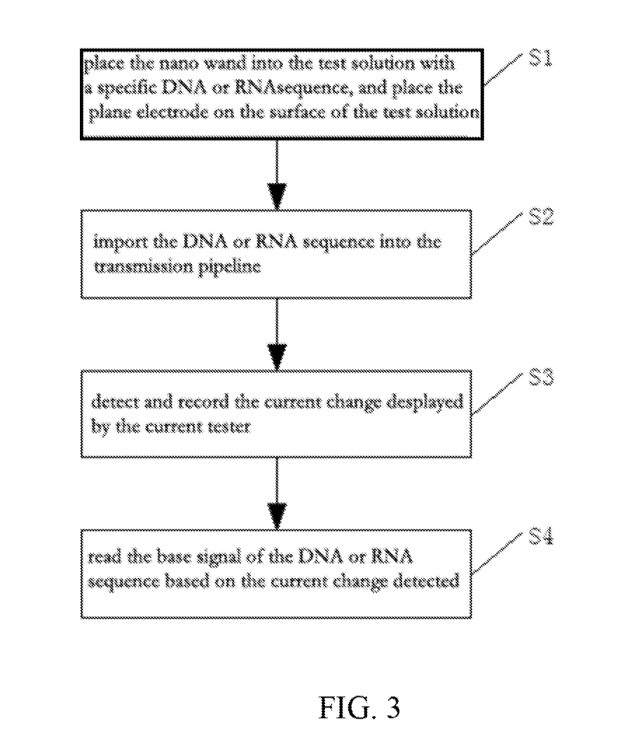

FIG. 3 depicts the flow diagram of a tunnel recognition technology-based nano-detection method. As show in FIG. 3, the said detection method consists of the following steps:

S1: Place the nano wand 1 used on the nano-detection device into the test solution with a specific DNA or RNA sequence 5, and place the plane electrode 2 on the surface of the said test solution;

S2: Import the DNA or RNA sequence into the transmission pipeline;

S3: Detect and record the current change displayed by the current tester 3;

S4: Read the base signal of the DNA or RNA sequence based on the current change detected

When the DNA or RNA sequence is imported through the transmission pipeline, it will give rise to change in the tunnel current between the nano wand electrode 11 and the plane electrode 2, and the corresponding base signal can be read via this current change.

Based on the quantum tunneling effect, when the DNA or RNA sequence passes through the transmission pipeline, the nano wand electrode will produce a potential difference, and give rise to change in the tunnel current between the nano wand electrode and the plane electrode on the surface of the test solution. Under the nanoscale, the current formed by the lateral movement of electrons driven by the tunneling effect (relative to the rear framework of DNA or RNA) will have an extremely high electric field intensity. For instance, when the bias voltage is 0.1V, a nano space of 1-2 nm will be able to provide an electric field of 10e6V/cm. An electric field of this intensity can very easily interact with bases to form dipoles. Under the action of such a high-intensity transverse electric field, single bases will be orderly arranged along the electrode direction. This effect can not only contribute to the orderly arrangement of bases and thus reduce the noise of the thermal structure, but also facilitate the movement of DNA and RNA through the transmission pipeline. In addition, by virtue of the frictional force between the bases and the electrode as a result of interactions, the movement rate of DNA or RNA can be controlled as well. Based on the above reasons, this detection method will see a significantly increased sequencing velocity than existing technologies.

To control the transmission velocities of DNA and RNA in step S2, in one example of the present invention, the transmission velocity of the said DNA or RNA sequence is changed through changing the said test solution's salt concentration gradient.

In one example of the present invention, the transmission velocity of the said DNA or RNA sequence is changed through changing the said test solution's electric field intensity.

In one example of the present invention, the transmission velocity of the said DNA or RNA sequence is changed through changing the said test solution's external pressure.

Readers should understand that, in the description of this specification, the representations by reference terms such as “one example”, “some examples”, “embodiment”, “specific embodiment” and “some embodiments” mean that the specific characteristics, structures, materials or features described by these examples or embodiments are contained in at least one example or embodiment of the present invention. In this specification, the schematic representation of the above terms does not have to target the same example or embodiment. Furthermore, the specific characteristics, structures, materials or features described can be combined with any or several examples or embodiments where appropriate. In addition, without causing contradictions, technical staff of this field may unite or combine different examples or embodiments described in this specification with their characteristics.

Although we have indicated and described the examples of the present invention above, what is understandable is that, the above examples are exemplary and cannot be interpreted as any limitation to the present invention. Ordinary technical staff of this field may make changes, modifications, replacements and transformations to the above examples within the scope of the present invention.

What is claimed is:

1. A tunnel recognition technology-based nano-detection device, wherein, the said detection device consists of nano wand, plane electrode, active power and current tester; the said nano wand is made through double-bore quartz tube drawing; one bore of the said double-bore quartz tube is made into an electrode through electrode material filling, while the other bore serves as the transmission pipeline; one end of the said electrode is connected to one end of the current tester, while the other end of the said electrode is connected to the active power; electrical connection is provided between the said plane electrode and the other end of the said current tester.

2. A tunnel recognition technology-based nano-detection device according to claim 1, wherein, the two bores of the said double-bore quartz tube have the same inner diameter ranging between 10 and 100 nm, and the bore edge distance of the said two bores ranges between 1 and 10 nm.

3. A tunnel recognition technology-based nano-detection device according to claim 2, wherein, the said inner diameter is 50 nm, and the said bore edge distance is 2.5 nm.

4. A tunnel recognition technology-based nano-detection device according to any of claims 1-3, wherein, the said electrode is a carbon electrode.

5. A tunnel recognition technology-based nano-detection device according to claim 4, wherein, the said electrode and the said plane electrode surface are decorated with recognition molecules, and the said recognition molecules are connected to the said electrode and the said plane electrode surface via trimethyl.

6. A tunnel recognition technology-based nano-detection device according to claim 4, wherein, the said carbon electrode is replaced by a gold electrode or palladium electrode, or the said carbon electrode surface is electroplated with a layer of gold or palladium film.

7. A tunnel recognition technology-based nano-detection device according to claim 6, wherein, the said electrode and the said plane electrode surface are decorated with recognition molecules, and the said recognition molecules are connected to the said electrode and the said plane electrode surface via mercapto group.

8. A tunnel recognition technology-based nano-detection device according to claim 1, wherein, the said current tester is connected to a host computer or other detection systems.

9. A tunnel recognition technology-based nano-detection method, wherein, the said detection method consists of the following steps:
Place the said nano wand used on the said tunnel recognition technology-based nano-detection device according to claim 1 into the test solution with a specific DNA or RNA sequence, place the said plane electrode on the surface of the said test solution;
Import the said DNA or RNA sequence into the said transmission pipeline;
Detect and record the current change displayed by the said current tester;
Read the base signal of the said DNA or RNA sequence based on the said current change detected.

10. A tunnel recognition technology-based nano-detection method according to claim 9, wherein, in the said step of importing the said DNA or RNA sequence into the said transmission pipeline, the transmission velocity of the said DNA or RNA sequence is changed through changing the said test solution's salt concentration gradient, electric field and external pressure.
